SEQUENCE LISTING (11 sequences)

SEQ ID NO: 1 - protein, 45 aa, Homo sapiens
TTTPAPRPPTPAPTIASQPLSLRPEACRPAAGGAVHTRGLDFACD

SEQ ID NO: 2 - protein, 14 aa, Homo sapiens
GKPIPNPLLGLDST

SEQ ID NO: 3 - protein, 14 aa, Homo sapiens
GKPIPNPLLGLDST

SEQ ID NO: 4 - DNA, 225 bp, Homo sapiens
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 5 - protein, 214 aa, Homo sapiens
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 237 aa, Homo sapiens
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKRTVAAPSVFIFPPSDEQLKSGTASVVCLLNNFYPREAKVQWKVDNALQSGNSQESVTEQDSGGGGSNYHLENEVARLKKLGGGGSDSTYSLSSTLTLSKADYEKHKVYACEVTHQGLSSPVTKSFNRGEC

SEQ ID NO: 7 - protein, 223 aa, Homo sapiens
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 8 - protein, 245 aa, Homo sapiens
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSGGGGSNYHLENEVARLKKLGGGGSLYSLSSVVTVPSSSLGTQTYICNVNHKPSNTKVDKKVEPKSC

SEQ ID NO: 9 - protein, 9 aa, Homo sapiens
RKKRRQRRR

SEQ ID NO: 10 - protein, 13 aa, Homo sapiens
GRKKRRQRRRPPQ

SEQ ID NO: 11 - protein, 26 aa, Homo sapiens
VQRKRQKLMPSKKKKIKVGRKRKKRT

(19) United States
(12) Patent Application Publication
(10) Pub. No.: US 2022/0364102 A1
(43) Pub. Date: Nov. 17, 2022

(54) COMPOSITIONS AND METHODS OF REPROGRAMMING T CELLS TO TREAT DISEASE

(71) Applicant: MedicaMetrix, Inc., Lowell, MA (US)

(72) Inventor: Vinoth Kumar Lakshmanan, Tamil Nadu (IN)

(21) Appl. No.: 17/743,069

(22) Filed: May 12, 2022

Related U.S. Application Data
(60) Provisional application No. 63/188,782, filed on May 14, 2021.

(51) Int. Cl. C12N 15/52; C07K 14/54; C12N 9/22; C12N 15/10

(52) U.S. Cl. CPC C12N 15/52; C07K 14/5434; C12N 9/22; C12N 15/102; A61K 38/00

(57) ABSTRACT

Disclosed herein are formulations and methods for the treatment of disease. The formulations and methods allow for the reprogramming of immune cells in a subject, particularly the T cells of a subject. The formulations are nanoparticles that have an interior and exterior in which the interior includes DNA molecules that encode genes for reprogramming T cells. The exterior of the nanoparticles targets the T cells to provide the DNA to the T cells.

### CROSS-REFERENCE TO RELATED APPLICATION

This application claims priority to, and the benefit of, U.S. Provisional Application No. 63/188,782, filed on May 14, 2021, the content of which is incorporated by reference herein in its entirety.

### SEQUENCE LISTING

This application contains a sequence listing which has been submitted in ASCII format via EFS-Web and is hereby incorporated by reference in its entirety. The ASCII-formatted sequence listing, created on May 12, 2022, is named “SQLIST-MEDM-004-01US”, and is 10214 bytes in size.

### FIELD

This disclosure relates generally to the field of medicine, and more specifically to treating cancer and other diseases.

### BACKGROUND

Cancer is a significant healthcare issue for the world's population. As an example, liver cancer in adult men is the fifth most frequently diagnosed cancer worldwide and is the second leading cause of cancer-related death in the world. Numerous therapeutic strategies have been employed to effectively treat cancer. Traditional therapeutic approaches have revolved around the use of chemotherapy and radiation therapy.

Chemotherapy involves administration of one or more anti-cancer drugs and/or other agents to a cancer patient by various methods. Chemotherapeutic drugs typically work by impairing mitosis, effectively targeting fast-dividing cells. However, other fast dividing cells such as those responsible for hair growth and for replacement of the intestinal lining are also affected. Because chemotherapy affects cell division, both normal and cancerous cells are susceptible to the cytotoxic effects of chemotherapeutic agents.

Cancer treatment can also involve surgical intervention and radiation therapy. Surgical interventions remove malignant tumors. Surgical intervention however has many drawbacks including significant recovery time and inapplicability to removal of tumors from certain locations. In addition, surgical interventions often do not remove the entire tumor and require other treatment regimes.

Radiation therapy also has been used for decades to treat cancer. This treatment requires exposing a patient to high-energy radiation, including x-rays, gamma rays, and neutrons. This type of therapy includes without limitation external-beam therapy, internal radiation therapy, implant radiation, brachytherapy, systemic radiation therapy, and radiotherapy. External beam radiation may include three-dimensional conformal radiation therapy, intensity modulated radiation therapy, and conformal proton beam radiation therapy. In practice it is difficult to shield the nearby normal tissue from the cytotoxic effects of the radiation and still deliver a therapeutic dose. An additional complication of radiation is the induction of radiation resistant cells during the course of treatment. Thus, even the best radiotherapeutic techniques often result in incomplete tumor reduction and subsequent recurrence.

More recently, immunotherapeutic approaches have been employed to harness the power of the host's immune system to treat cancer. For example, strategies have been employed to target cancer-associated antigens with host-based T cells that specifically recognize such antigens. For example, a recent approach has focused on the development and use of chimeric antigen receptor (CAR) T cells (also known as CAR-T cells). Possible side effects associated with CAR-T cell therapy include chemokine-release syndrome, B cell aplasia, and tumor lysis syndrome. Despite the development of these approaches, cancer remains a significant healthcare issue.

### SUMMARY

Disclosed herein are methods of treating disease, as well as vectors, nanoparticles, and cellular mechanisms for the treatment of diseases such as cancer. In particular, disclosed herein is a reprogrammed switchable TRUCK-T cell method of treating disease and the compositions necessary to perform the method. As disclosed, TRUCK-T cells are T cells Redirected for antigen-Unrestricted Cytokine-initiated Killing. These T cells can be turned on and off depending on a signal received by the T cells.

Aspects disclosed herein disclose a nanoparticle comprising interior portion comprising one or more DNA molecules and a polymer bound to a protein. In particular embodiments, the protein comprises a first amino acid sequence directing the protein to the nucleus of a cell and a second sequence, wherein the one or more DNA molecules are associated with the polymer by way of opposing charges.

In certain embodiments, the interior portion has a net positive charge. In other embodiments, the protein further comprises a second sequence. In some embodiments, the second sequence is a TAT sequence or MLS sequence.

In particular embodiments, the polymer is selected from the group consisting of poly-β-amino ester, polyethylenimine, pentaethylenehexamine, polyethylene glycol, polyvinyl alcohol, poly(lactide-co-glycolide, poloxamer, poly(N-vinylpyrrolidone, gelatin, human albumin, and starch. In more particular embodiments, the polymer is poly-β-amino ester. In still more particular embodiments, the polymer is polyethylenimine.

In some embodiments, the DNA is a minicircle plasmid. In other embodiments, the DNA molecules comprise two or more genes. In still other embodiments, a first gene of the two or more genes encodes a CAR gene. In yet other embodiments, a second gene of the two or more genes encodes an IL-12 protein. In more embodiments, a third gene of the two or more genes encodes an Cas9 endonuclease. In certain embodiments, the Cas9 endonuclease is modified to cleave a DNA sequence in the PD1 gene to inactivate the PD1 gene. In particular embodiments, a fourth gene of the two or more genes encodes a second Cas9 endonuclease modified to cleave a DNA sequence in the CTL4A gene to inactivate the CTL4A gene. In very particular embodiments, a fifth gene of the two or more genes encodes a targeting component to target a cell.

In certain embodiments, the targeting component is selected from the group consisting of recombinant antibodies, monoclonal antibodies, Fab fragments, Fab2 fragments, single chain variable fragments, diabodies, and receptor ligands. In other embodiments, the targeting component is a Fab fragment. In still other embodiments, the one or more DNA molecules comprise a promoter sequence controlling the expression of two or more genes. In more embodiments, the one or more DNA molecules comprise a reporter molecule under the control of a promoter. In still more embodiments, the one or more DNA molecules comprises a WPRE sequence. In yet more embodiments, the one or more DNA molecules comprises an MAR sequence. In yet other embodiments, the one or more DNA molecules comprises polyadenylation sequence.

In particular embodiments, the CAR gene encodes a molecule comprising a transmembrane domain, one or more cytoplasmic domains, and a targeting ligand. In more particular embodiments, the targeting ligand is a variable heavy chain and a variable light chain of a Fab fragment. In yet more particular embodiments, the targeting component is a Fab fragment comprising a tag and wherein the one or more DNA molecules comprise a first gene that encodes a CAR gene. In still more particular embodiments, the CAR gene encodes a molecule comprising a transmembrane domain, one or more cytoplasmic domains, and a targeting ligand and wherein the targeting ligand binds to the tag.

In other embodiments, the tag is a V5 tag, and the targeting ligand is an anti-V5 Fab fragment. In still other embodiments, the molecule further comprises a CD3-theta domain. Some embodiments further comprise an exterior. In more embodiments, the exterior comprises a polymer and a targeting group. In particular embodiments, the polymer is polyglutamic acid and the targeting group is selected from the group consisting of recombinant antibodies, monoclonal antibodies, Fab fragments, Fab2 fragments, single chain variable fragments, diabodies, and receptor ligands. In more particular embodiments, the targeting group is a Fab fragment.

In other embodiments, the targeting group is an anti-CD3 Fab fragment, anti-CD19 Fab fragment, or anti-CD22 Fab fragment. In yet other embodiments, the nanoparticle is lyophilized. In still other embodiments, the exterior has a net negative charge. In further embodiments, the second sequence allows the one or more DNA molecules to move through a membrane. In still further embodiments, the second sequence allows the one or more DNA molecules to localize to the nucleus of a cell.

Aspects disclosed herein also disclose a method of treating a disease in a subject. The method comprises administering an effective amount of a nanoparticle to the subject, the nanoparticle comprising one or more DNA molecules and a polymer bound to a protein, the protein comprising a first amino acid sequence directing the protein to the nucleus of a cell and a second sequence, wherein the one or more DNA molecules are associated with the polymer by way of opposing charges, wherein the nanoparticles reprogram a plurality of T cells in the subject; through the T cells, targeting a plurality of cells causing the disease in the subject, wherein the T cells instigate the death of the plurality of cells causing the disease, thereby treating the disease.

In certain embodiments, the interior portion has a net positive charge. Some embodiments allow for the interior portion to have a net positive charge. In other embodiments, the protein further comprises a second sequence. In some embodiments, the second sequence is a TAT sequence or MLS sequence.

In particular embodiments, the polymer is selected from the group consisting of poly-β-amino ester, polyethylenimine, pentaethylenehexamine, polyethylene glycol, polyvinyl alcohol, poly(lactide-co-glycolide, poloxamer, poly(N-vinylpyrrolidone, gelatin, human albumin, and starch. In more particular embodiments, the polymer is poly-β-amino ester. In still more particular embodiments, the polymer is polyethylenimine.

In some embodiments, the DNA is a minicircle plasmid. In other embodiments, the DNA molecules comprise two or more genes. In still other embodiments, a first gene of the two or more genes encodes a CAR gene. In yet other embodiments, a second gene of the two or more genes encodes an IL-12 protein. In more embodiments, a third gene of the two or more genes encodes an Cas9 endonuclease. In certain embodiments, the Cas9 endonuclease is modified to cleave a DNA sequence in the PD1 gene to inactivate the PD1 gene. In particular embodiments, a fourth gene of the two or more genes encodes a second Cas9 endonuclease modified to cleave a DNA sequence in the CTL4A gene to inactivate the CTL4A gene. In very particular embodiments, a fifth gene of the two or more genes encodes a targeting component to target a cell.

In certain embodiments, the targeting component is selected from the group consisting of recombinant antibodies, monoclonal antibodies, Fab fragments, Fab2 fragments, single chain variable fragments, diabodies, and receptor ligands. In other embodiments, the targeting component is a Fab fragment. In still other embodiments, the one or more DNA molecules comprise a promoter sequence controlling the expression of two or more genes. In more embodiments, the one or more DNA molecules comprise a reporter molecule under the control of a promoter. In still more embodiments, the one or more DNA molecules comprises a WPRE sequence. In yet more embodiments, the one or more DNA molecules comprises an MAR sequence. In yet other embodiments, the one or more DNA molecules comprises polyadenylation sequence.

In particular embodiments, the CAR gene encodes a molecule comprising a transmembrane domain, one or more cytoplasmic domains, and a targeting ligand. In more particular embodiments, the targeting ligand is a variable heavy chain and a variable light chain of a Fab fragment. In yet more particular embodiments, the targeting component is a Fab fragment comprising a tag and wherein the one or more DNA molecules comprise a first gene that encodes a CAR gene. In still more particular embodiments, the CAR gene encodes a molecule comprising a transmembrane domain, one or more cytoplasmic domains, and a targeting ligand and wherein the targeting ligand binds to the tag.

In other embodiments, the tag is a V5 tag, and the targeting ligand is an anti-V5 Fab fragment. In still other embodiments, the molecule further comprises a CD3-theta domain. Some embodiments further comprise an exterior. In more embodiments, the exterior comprises a polymer and a targeting group. In particular embodiments, the polymer is polyglutamic acid and the targeting group is selected from the group consisting of recombinant antibodies, monoclonal antibodies, Fab fragments, Fab2 fragments, single chain variable fragments, diabodies, and receptor ligands. In more particular embodiments, the targeting group is a Fab fragment.

In other embodiments, the targeting group is an anti-CD3 Fab fragment, anti-CD19 Fab fragment, or anti-CD22 Fab fragment. In yet other embodiments, the nanoparticle is lyophilized. In still other embodiments, the exterior has a net negative charge. In further embodiments, the second sequence allows the one or more DNA molecules to move through a membrane. In still further embodiments, the second sequence allows the one or more DNA molecules to localize to the nucleus of a cell.

In certain embodiments, the disease is a cancer selected from the group consisting of myeloblastic leukemia, promyelocytic leukemia, myelomonocytic leukemia, monocytic leukemia, erythroleukemia leukemia, chronic myelocytic leukemia, chronic lymphocytic leukemia, lymphoma, fibrosarcoma, mycosarcoma, liposarcoma, chondrosarcoma, osteogenic sarcoma, angiosarcoma, endotheliosarcoma, colon carcinoma, pancreatic cancer, breast cancer, ovarian cancer, prostate cancer, squamous cell carcinoma, basal cell carcinoma, adenocarcinoma, renal cell carcinoma, hepatoma, penile cancer, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia.

In other embodiments, the disease is caused by a pathogen selected from the group consisting of yeast, Streptococcus pyogenes, Streptococcus pneumoniae, Neisseria gonorrhoeae, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromatis, Staphylococcus aureus, Vibrio cholerae, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazekii, Rickettsia tsutsugumushi, Chlamydia spp., and Helicobacter pylori.

In other embodiments, the effective amount of the nanoparticle is from about 100 μg to about 2,000 μg.

### BRIEF DESCRIPTION OF THE FIGURES

The invention will be more fully understood from the following detailed description taken in conjunction with the accompanying drawings, in which:

FIG. 1 is a pictorial representation showing two plasmids used in embodiments disclosed herein;

FIG. 2 is a pictorial representation showing a CAR protein;

FIG. 3 is a pictorial representation showing a nanoparticle in an embodiment disclosed herein;

FIG. 4 is a pictorial representation showing a nanoparticle in an embodiment disclosed herein;

FIG. 5 shows a mechanism of expressing a transposed gene within a genome of a cell;

FIG. 6 shows a gene cassette comprising a CAR gene and the gene product expressed from the gene;

FIG. 7 shows a Fab fragment directed against PSA;

FIG. 8 shows a gene cassette of one embodiment of the disclosed nanoparticles;

FIG. 9 shows a gene cassette of one embodiment of the disclosed nanoparticles;

FIG. 10 shows a gene cassette of one embodiment of the disclosed nanoparticles;

FIG. 11 shows a gene cassette of one embodiment of the disclosed nanoparticles;

FIG. 12 shows a mechanism of a reprogrammed T cell killing a target cell such as a tumor cell; and

FIG. 13 shows a mechanism of a reprogrammed T cell killing a target cell such as a tumor cell.

### DETAILED DESCRIPTION

Chimeric antigen receptor (“CAR”) T-cell (“CAR-T”) therapy has emerged as a novel therapeutic for cancer therapy. The technology involves in vitro engineering of T cells derived from a patient such that the T cells have high affinity receptors targeted to a specific tumor antigen. In the last few years, the use of CAR-T cell therapy has been shown to have promise with 80% reduction in remission rates for hematologic cancer, especially—non-Hodgkin lymphomas, such as large B cell lymphoma and acute lymphoblastic leukemia (ALL). Recently in terms of efficacy, anti-CD19 CAR therapy, or UCART19, has been effective in treating relapsed/refractory hematologic cancer.

CARs are made up of two major domains: the ectodomain and the endodomain. The endodomain consists of portions of signaling molecules that are important for activation of T cells. This domain has evolved considerably since the introduction of CARs. The first generation of CARs contained a portion of a molecule called CD3 that is responsible for signaling that the receptor has been bound from the outside. Second generation CARs included a portion of another protein, CD28, which is a costimulatory molecule that enhances the signal from the receptor. As research on CARs and their components continued, the endodomain underwent further refinement. Third generation CARs currently contain additional signaling domains that enhance the signal and/or survival of T cells. Aspects disclosed herein further allow for nanoparticles to deliver the 4th generation of CAR T cell engineering and provide an outlook on novel CAR formats designed gene cassette.

In some embodiments, the disclosed CAR gene cassettes include an antigen recognition domain where the antigen-binding properties of a CAR which usually consists of a single-chain variable fragment (scFv). In particular embodiments, scFv is generated through linking the variable light (VL) and variable heavy (VH) regions of a monoclonal antibody by a short linker. The configuration in which VL is followed by the linker then by VH more closely mimics the natural antibody design and would therefore be superior. However, empirical testing has revealed that both VL-linker-VH and VH-linker-VL configurations can function properly. There are different linker molecules have been successfully utilized in designing scFvs. Currently, the majority of linkers used in CART cells encompass some variation of a polypeptide based on glycine (Gly) and serine (Ser) repeats. For example, the (Gly4Ser)3-linker consists of three repeats of the pentapeptide Gly-Gly-Gly-Gly-Ser. The use of these residues is intended to confer flexibility and minimize the risk for interference of the linker with the proper folding and function of the connected CAR protein domains. In our switchable CAR protein design, heavy chain (VH) and light chain (VL) from the anti-V5 antibody facilitate through tonic signals with optimal linker length is within the range of 15-20 amino acids by utilizing (Gly4Ser)3 or (Gly4Ser)4 linkers. The disclosed linkers confer flexibility to the antibody for optimal target recognition spacer length can be tuned to optimize the immunological synapse distance, unmodified IgG spacers may bind to Fc-receptor, spacers may induce tonic CAR signaling.

CD8α and CD28 hinge regions can be utilized (Maude, N. Engl. J. Med. 2014). Incorporating hinge (spacer) of CD28 of transmembrane domain CD28 can increase CAR surface expression linking extracellular domain to the cytoplasmic domain CD28 along with costimulatory domains region such as 4-1BB and CD3ζ confers potent and short-lived effector functions of 4-1BB increases CART cell persistence. Moreover, these costimulatory domains may facilitate tonic signaling under certain circumstances and participates in activating both CD8 T cells and CD4 T cells. When induced by a signal, the ITAM region (not shown) of the CD3-theta becomes phosphorylated and capable of binding ZAP70, a kinase that induces a signaling cascade that activates the T cell.

The gene cassette in some embodiments, includes a Matrix attachment region (MAR) that will contain 1-68 AT rich core region act as epigenetic regulatory sequences that increase gene expression, and the Cytomegalovirus (CMV) promoter, the eukaryotic translation elongation factor 1 α (EF-1α) (gene symbol EEF1A1) promoter sequence (470-1653) will increase transfection efficiency with maintenance of transgene expression, stability, and copy number. The disclosed CAR cassette plasmid design can contain the woodchuck hepatitis post-translational regulatory element (WPRE) nucleotides (1093 to 1684) (GeneBank accession No.J04514), will be inserted between reporter gene such as Enhanced Green Fluorescent Protein (EGFP) and a polyadenylation sequence such as the BGH-polyA sequence (3544-3771) (see, e.g., Utratna and O'Byrne (2014) Methods Mol Biol. 1157:233-47). The Orf of our CAR design will be C-terminally fused to the minicircle DNA sequence coding for EGFP to allow for a co-translation of CAR and EGFP from one mRNA, both reading frames will be separated by a 2A ‘cleavage’ site derived from Thosea asigna virus that will induces a ribosomal ‘skip’ from one codon to the next without formation of a peptide bond (see Koristka Can Imm Immunother 1401-1415).

In certain embodiments, a minicircle DNA comprising the CAR gene cassette further comprises a gene cassette for targeting the T cell to a particular cell type. Our Fab fragment for target such as (prostate-specific antigen (PSA), prostate specific membrane antigen (PSMA), prostate cancer antigen (PCA3) and CD24.)

The present disclosure provides for compositions that can be administered to a subject to alter the function of one or more T cells. As disclosed herein, vectors for transformation of eukaryotic cells are produced using known methods. An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

Aspects of the present invention include delivery of nanoparticles comprising plasmids to T cells. In certain embodiments, the plasmids comprise genes-of-interest to reprogram T cells to control activation of the T cell and to direct the T cell to attack a particular cell such as a cancer cell to induce cell death. When reprogrammed, T cells can be used to treat diseases disclosed herein.

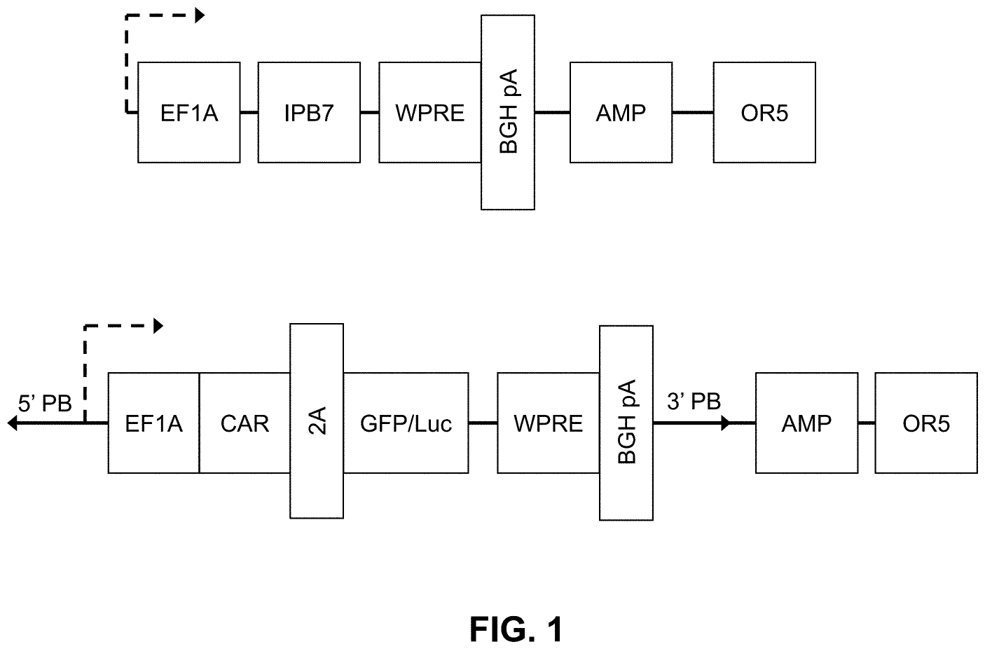

Aspects disclosed herein include methods of expressing genes-of-interest in T cells. One method of expressing genes-of-interest in T cells includes using plasmid sequences comprising a multitude of sequences to enhance protein expression in a eukaryotic cell (see FIG. 1). In certain embodiments, the plasmid DNA is minicircle DNA. For instance, the sequences include a promoter. Such promoters may be the EF-1 promoter, SV40 promoter, cytomegalovirus promoter (CMV), CAG promoter, PGK promoter, TRE promoter, U6 promoter, or Rous Sarcoma Virus (RSV). In certain embodiments, the promoter is an EF-1α promoter. In addition, the sequences include a gene expression cassette that allows insertion of a gene-of-interest in proper position downstream of the promoter sequences. Downstream from the gene-of-interest can be a Woodchuck Hepatitis Virus (WHP) Posttranscriptional Regulatory Element (WPRE), which is used to enhance the expression of the gene-of-interest. In some embodiments, there is a polyadenylation sequence downstream of the gene-of-interest for eukaryotic expression. One example of a polyadenylation sequence is Bovine Growth Hormone polyadenylation, shown as BGH-PA in FIG. 1.

In some embodiments, the genes-of-interest are under the control of an inducible promoter. Inducible promoters allow for turning gene expression on and off under the influence of a signal. Inducible promoters also allow for the control of cellular functions. As in the presently disclosed techniques, inducible promoters would turn the T cell activities on and off based on the signal. An example of inducible eukaryotic expression is a CMV promoter under the control of a tetracycline resistance operon. Any one of the genes-of-interest disclosed herein can be regulated by an inducible promoter according to methods disclosed herein.

The plasmid also comprises sequences that allow for selection of plasmids during bacterial production of the plasmid sequences. In certain embodiments, the plasmid sequences comprise an ampicillin resistance gene (AMP) and an origin of replication (ORI). Plasmids can also comprise reporter genes such as GFP/Luciferase (see FIG. 1, GFP/Luc). Such plasmids may include viral promoter sequences in addition to eukaryotic promoter sequences to ensure co-translation of reporter sequences. FIG. 1 shows one embodiment in which the promoter is the viral 2A promoter. When co-expressed with the gene of interest, the reporter sequence is cleaved from the gene of interest and can be identified using standard techniques.

FIG. 1 shows two expression plasmids that can be co-delivered to a T cell. The co-delivered plasmids of FIG. 1 comprise one plasmid expressing iPB7, which mediates increased expression in vivo. iPB7 is the iPB gene with seven amino acid substitutions and has an amino acid sequence shown in SEQ ID NO. 1. iPB7 facilitates transposition of a gene-of-interest into a eukaryotic cell's genome. Any transposase gene can be used in a co-delivered plasmid.

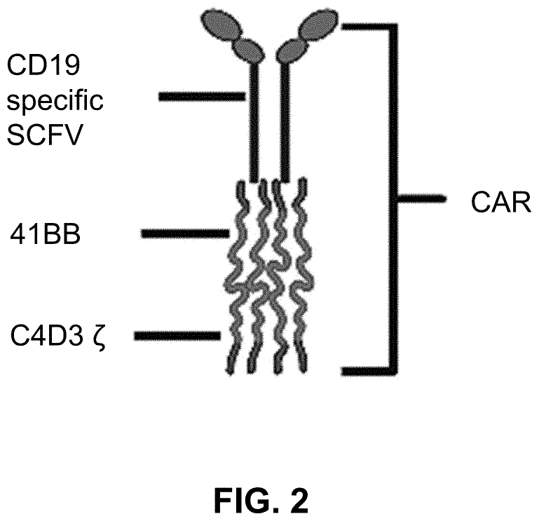

FIG. 1 also shows a second plasmid comprising the gene of interest—CAR. In certain embodiments, CAR encodes a m194-1BBz CAR shown in FIG. 2. m194-1BBz CAR comprises a CD19-specific scFv fused to the 4-1BB gene, which encodes CD137. CD137 is an inducible costimulatory receptor (SEQ ID NO. 2). m194-1BBz CAR also encodes the gene encoding T-cell surface glycoprotein CD3-zeta chain (FIG. 2, also shown SEQ ID NO. 3). In certain embodiments, the CAR gene is expressed with the GFP/Luc gene that is cleaved from the CAR gene product. The GFP/Luc gene allows for identification of cells that have been transformed by the co-delivery of plasmids.

In embodiments utilizing minicircle DNA, methods of producing minicircle DNA are known (see, e.g., Kay et al. 2010 Nat Biotechnol. 28(12): 1287-1289). One method for making minicircle DNA is to clone a gene-of-interest into a plasmid comprising bacterial sequences such as an origin, a resistance gene sequence, as well as eukaryotic promoters and polyadenylation sequences. The plasmid is transformed into a competent E. coli such as ZYC10P3S2T. The competent E. coli is induced with arabinose that switches on θC31 integrase and Sce-I endonuclease genes such that the plasmid is cleaved into two smaller plasmids. The plasmid comprising the bacterial sequences is degraded while the “minicircle DNA” remains. The minicircle DNA is isolated and purified.

A multitude of expression vectors can be used in the disclosed nanoparticles and methods. Examples of suitable expression vectors include pMC.BESPX-MCS1, pMC.BESPX-MCS2, pMC.CMV-MCS-sv40polyA, pMC.EF1-MCS-sv40polyA, pMC.CMV-MCS-EF1-GFP-sv40polyA, pMC.CMV-MCS-EF1-GFP-sv40polyA, pMC.EF1.MCS-IRES-GFP-sv40polyA, pMC.EF1-MCS-IRES-RFP-sv40polyA, and pMC.shRNA vectors. These vectors can be obtained commercially from System Biosystems, LLC (Embarcadero Way, Palo Alto, Calif.). Another example of vectors that be used in the disclosed methods is the GeneArt® CRISPR Nuclease Vector available from Thermo Fisher Scientific (St. Louis, Mo.). In yet another example of a vector acceptable in the disclosed methods, the pEF-DEST51 vector is available from Thermo Fisher Scientific (St. Louis, Mo.).

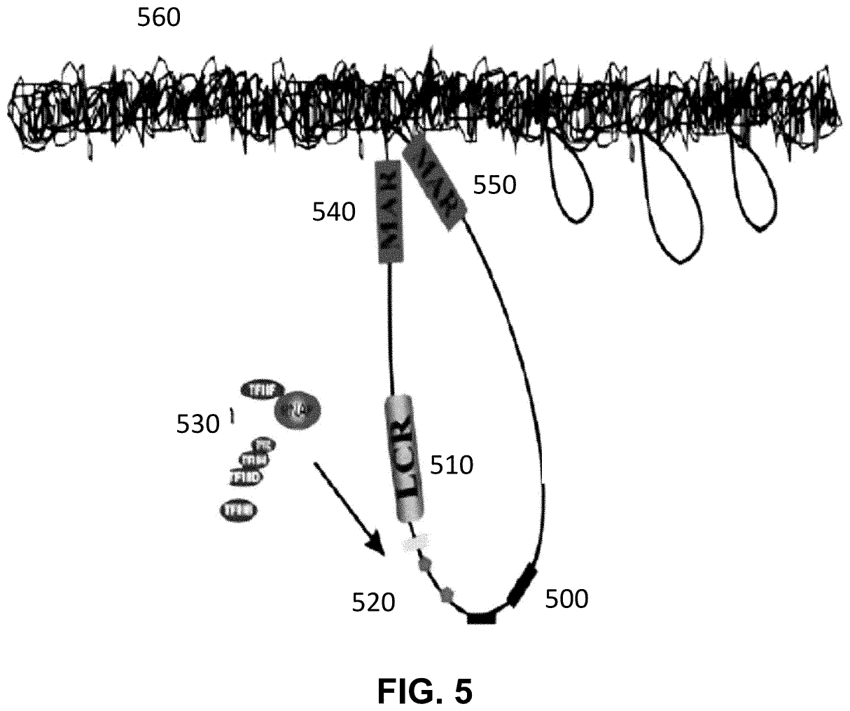

One of ordinary skill in the art would understand that co-expression of CAR and iPB7 will lead to incorporation of CAR into the genome of the cells that uptake the plasmids. Transposition of genes allows for stable expression of CAR gene during the life cycle of the cell. Once the genes are transposed into the genome, they can be stably expressed under the control of sequences that are transposed with the genes-of-interest. FIG. 5 shows one such mechanism. The transposed gene 500 is under the control of a locus control region 510 and transcription recruitment sites 520 that recruit transcription machinery 530 to control transcription. The transposed gene 500 is transposed with a Matrix Attachment Regions (MAR) 540 and 550 that allows for looping of the transposed gene 500 out of the heterochromatin 560 of the cell. This increases the expression of the gene 500. Such techniques are known (see, e.g., Papadakis, et al. (2004) Current Gene Therapy, 4(1):89-113).

Aspects of the disclosed compositions comprise formulations for delivery of the plasmids to T cells in a subject. The formulations comprise nanoparticles for delivery of plasmid DNA to a subject. The nanoparticles target T cells in a subject and transform T cells in the subject with one or more of the plasmids disclosed herein.

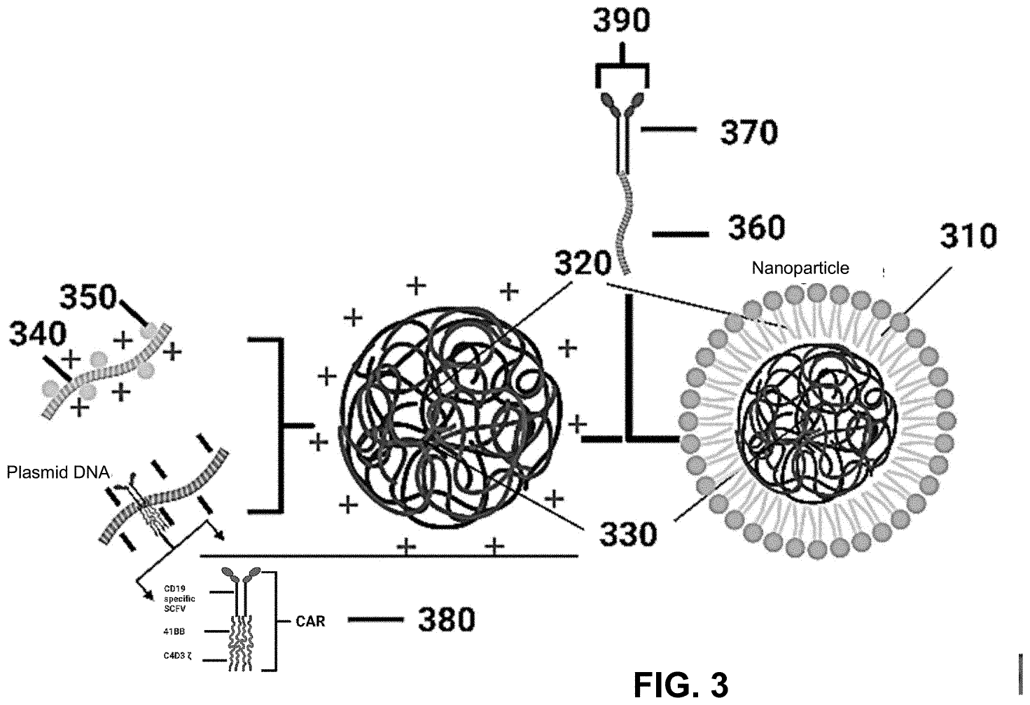

The nanoparticles disclosed herein have an interior 310 and exterior 320, as shown in FIG. 3. The interior of the nanoparticle can comprise plasmid DNA molecules. In certain embodiments, the nanoparticles comprise two or more plasmids 330 expressing a gene encoding a transposase and a gene cassette to express a CAR gene product 380. It is understood that a single plasmid can express multiple genes-of-interest such as a gene encoding a transposase and a gene cassette to express a CAR gene product, as well as other genes.

The two or more plasmids can also express a Cas9 gene to target particular genes. Cas9 (CRISPR associated protein 9, formerly called Cas5, Csn1, or Csx12), a 160 kD protein, is an RNA-guided DNA endonuclease (see, e.g., Deltcheva et al. (2011). Nature. 471 (7340): 602-607.). Cas9 can be modified to cleave particular sequences to inactivate a single gene or to allow insertion of novel sequences into a gene. In gene engineering, Cas9's primary function is to change the genome of a cell. In particular embodiments, Cas9 is directed to eliminate the function of programmed death 1 (PD1) and CTL4A. PD1 and CTL4A are involved in removing the T cell from circulation and downregulating immune function, respectively. By eliminating this functionality, the reprogrammed T cell cannot be downregulated by a target cell.

Aspects of the disclosed methods and formulations comprise minicircle DNA comprising genes-of-interest expressing targeting genes. In the embodiments, the targeting genes target the reprogrammed T cells to target the reprogrammed T cells to a cell for the purpose of killing the cell. Targeting genes can be induced to produce proteins that target cell surface receptors or other structures on the cell surface. Examples of such proteins include recombinant antibodies, monoclonal antibodies, Fab fragments, Fab2 fragments, single chain variable fragments, diabodies, and receptor ligands. In particular embodiments, the gene-of-interest encodes a Fab or Fab2 fragment. In other embodiments, the gene-of-interest encodes a ligand.

The interior 310 further comprises a polymer 340 conjugated with a peptide 350. The polymer 340 can be a polymer that permits conjugation with a peptide. Examples of polymers can be poly-β-amino ester (PBAE), polyethylenimine, pentaethylenehexamine, polyethylene glycol, polyvinyl alcohol, poly(lactide-co-glycolide, poloxamer, poly(N-vinylpyrrolidone, gelatin, human albumin, and starch. In some embodiments, the polymer 340 is PBAE-447. In particular embodiments, the peptide 350 provides a mechanism to translocate the DNA to the nucleus of the cell and is cationic to allow association of the polymer-peptide complex with the plasmid DNA. In very particular embodiments, the peptide 350 comprises SEQ ID NO. 4 that includes a microtubule-associated sequence (MTAS) and a nuclear localization sequence (“NLS”). Without being held to any particular theory, the peptide 350 comprising an MTAS and an NLS associated with the plasmid DNA and localizes the DNA via the cellular machinery to the nucleus of the cell. Once in the nucleus, the transposase enzyme transposes the CAR gene cassette to the DNA of the T cell.

The exterior 320 can comprise polymer 360. Examples of polymers include polyglutamic acid, and other polymers rich in amino groups. In some embodiments, the exterior 320 comprises polyglutamic acid. The polymer 360 can be conjugated to a targeting group 370—together comprising targeting polymer 390. The targeting group 370 allows for targeting of the nanoparticles to the T cell. Examples of targeting ligands include anti-CD3, anti-CD19, and anti-CD22. One of ordinary skill in the art will understand that any cell surface antigens can be used to target T cells so long as the cell surface antigen allows for transport of the nanoparticle interior 310 into the T cell.

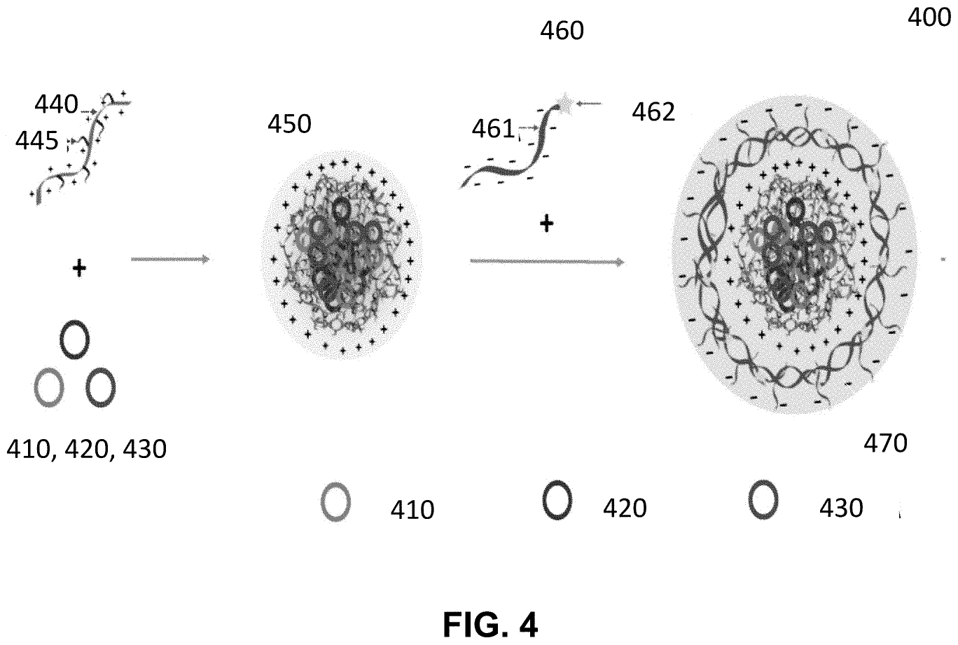

In FIG. 4, another embodiment of the nanoparticle is shown. In this embodiment, three minicircle plasmids are incorporated into the nanoparticle 400. DNA minicircles 410, 420, and 430 are associated with a polymer 440. In this embodiment, minicircle plasmids 410, 420, and 430 comprise genes-of-interest CAR, Cas9 to eliminate the function of PD1, and Cas9 to eliminate the function of CTL4A, respectively. In this embodiment, the reprogrammed T cells will not have the PD1 and CTL4A functionality. The polymer 440 is polyethylenimine decorated with a Tat-NLS peptide 445 (SEQ ID NO. 5) to provide a positive (+) charge to the polymer 440 and to direct via TAT gene sequences to the nucleus of the cell. The interior 450 of the nanoparticle 400 thus comprises 410, 420, and 430 associated with polymer 440. The interior 450 has a net positive (+) charge due to the peptide 445.

The interior 450 is then coated with multiple polymer complexes 460 comprising polyglutamic acids (PGA) 461 conjugated with a target ligand (anti-CD3) 462. The polymer complex 460 creates an exterior 470 surrounding the interior 450 to form the nanoparticle 400. The exterior 470 comprises a negative (−) charge. The nanoparticles 400 can be lyophilized for storage according to known techniques for administration to human subjects (see, e.g., world wide web at fda.gov/inspections-compliance-enforcement-and-criminal -investigations/inspection-guides/lyophilization-parenteral-793).

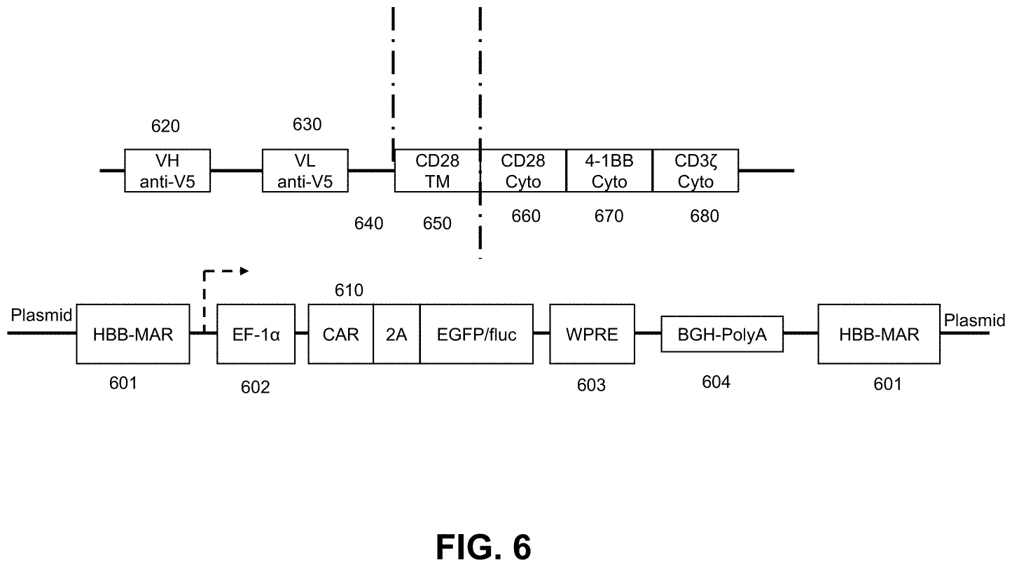

Aspects disclosed herein further allow for nanoparticles to deliver the CAR gene cassette. FIG. 6 shows an embodiment of the gene cassette 600 for the CAR gene 610. The CAR gene in this embodiment comprises a heavy chain 620 and light chain 630 from the anti-V5 antibody (SEQ ID NOS. 6 and 7, respectively). The CAR gene cassette 600 further includes a spacer sequence. In this embodiment, the spacer sequence is a CD28 spacer 640. The spacer 640 is fused to the CD28 transmembrane domain 650. As shown in FIG. 6, the CD28 transmembrane domain 650 is fused to the CD28 cytoplasmic domain 660, which is fused to the 4-1BB cytoplasmic domain 670. The 4-1BB cytoplasmic domain 670 is fused to the CD3-theta domain 680. CD3-theta participates in activating both CD8 T cells and CD4 T cells. When induced by a signal, the ITAM region (not shown) of the CD3-theta becomes phosphorylated and capable of binding ZAP70, a kinase that induces a signaling cascade that activates the T cell. The gene cassette in some embodiments, includes an MAR 601, the EF-1α promoter 602, a WPRE 603, and a polyadenylation sequence such as the BGH-polyA 604. In other embodiments, the CAR cassette includes a reporter gene such as Enhanced Green Fluorescent Protein (EGFP) 605 (see, e.g., Utratna and O'Byrne (2014) Methods Mol Biol. 1157:233-47).

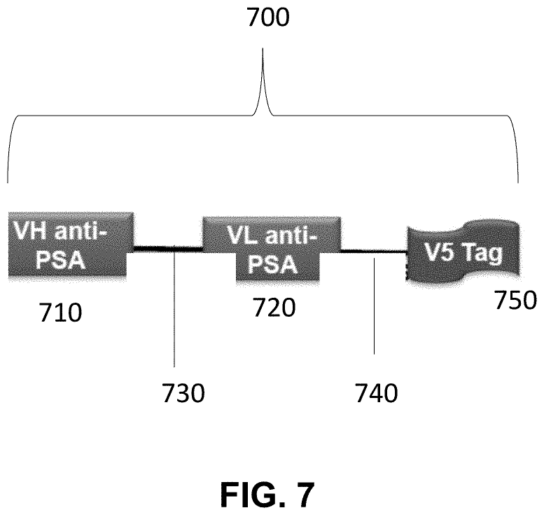

In certain embodiments, a minicircle DNA comprising the CAR gene cassette further comprises a gene cassette for targeting the T cell to a particular cell type. FIG. 7 shows a gene cassette 700 comprising a Fab fragment for targeting to the prostate-specific antigen (PSA). The sequences include the variable heavy chain 710 and variable light chain 720 (SEQ ID NOS. 8 and 9) for targeting to the PSA on the surface of prostate cancer cells. In this embodiment, the gene cassette 700 further includes a three GGGGS repeat sequence (SEQ ID NO. 10) 730 between the variable light chain 720 and variable heavy chain 710 as well as a GGGGS sequence 740 between the variable light chain sequence 720 and a V5 tag sequence 750. The V5 tag sequence 750 will allow for the anti-V5 targeting portion 710 and 720 of the CAR to bind and thereby identify a target cell.

The genetically engineered receptors allow CAR T-cells to recognize tumor cells with low antigen expression and cause direct lysis of tumor cells whereas classical monoclonal antibodies need a high density of tumor antigens to trigger the antibody dependent cellular cytotoxicity (ADCC) mediated by NK cells or complement cascade (Caruana et al. 2014; Gauthier and Yakoub-Agha 2017). Bispecific constructs have the same disadvantage as monoclonal antibodies, the newer formats can be made smaller than a classical immunoglobulin allowing better distribution but then usually have a reduced plasma half-life.

Disclosed herein are constructs of polyspecific antibodies such as tetraspecific antibodies that allow enhanced binding to target and in preclinical models have significantly enhanced anticancer activity compared to monoclonal antibodies. In certain embodiments, bispecific tetravalent molecules such as dual-variable-domain immunoglobulin (DVD-Ig) are produced by combining two target-binding monoclonal antibodies via naturally occurring linkers. Recently, targeting simultaneously with multiple antigens using polyspecific antibodies usually have two binding sites (bispecific), there are many new molecules with three or four binding sites. Methods of making tetra specific antibodies are disclosed in Castoldi et al., 2016 to make a tetravalent and tetraspecific antibody (1+1+1+1 antigen-binding valency). Tetraspecific antibodies offer multiple specificities with one or more affinity sites towards tumor antigens, and another one towards an activator on immune effectors (e.g., CD3 on T cells). So, the in the CART cells T cells with genetically engineered receptors that redirect them to a chosen tumor antigen (Runcie et al. Mol Med 2018).

The disclosed tetraspecific antibodies can simultaneously engage two or more different types of epitopes. In particular embodiments, the tetraspecific antibodies: 1) redirect specific polyclonal immune cells such as T cells and NK cells to tumor cells to enhance tumor killing; 2) simultaneously block two different pathways with unique or overlapping functions in pathogenesis; 3) potentially increase binding specificity by interacting with two different cell surface antigens instead of one; and 4) reduce cost in terms of development and production when compared to multiple single based antibodies used in combination therapy or compared to the production of CAR-T cells.

Aspects disclosed herein allow for Car/TRUCK can be directed to target cells by tetraspecific antibodies that act as a bridge between the target cell and CAR-T cell. The engineering of recombinant antibody-based dual switches that consist of a tumor antigen-specific Fab molecule engrafted with a peptide neo-epitope, which is bound exclusively by a peptide-specific switchable CAR-T cell.

Examples of antibodies that bridge to tumor cells include antibodies directed against antigens including alphafetoprotein, carcinoembryonic antigen (CEA), CA-125, epithelial tumor antigen (ETA), melanoma-associated antigen, RAS, p53, AR, PSA, PSMA, CD24, PCA3, BRCA1, BRCA2, CTLA-4, and PD1. It should be noted that so long as an antigen can induce an immune response, the presently disclosed techniques can be used to generate antibodies against the antigen and allow for targeting of CAR/TRUCK cells to the target cell.

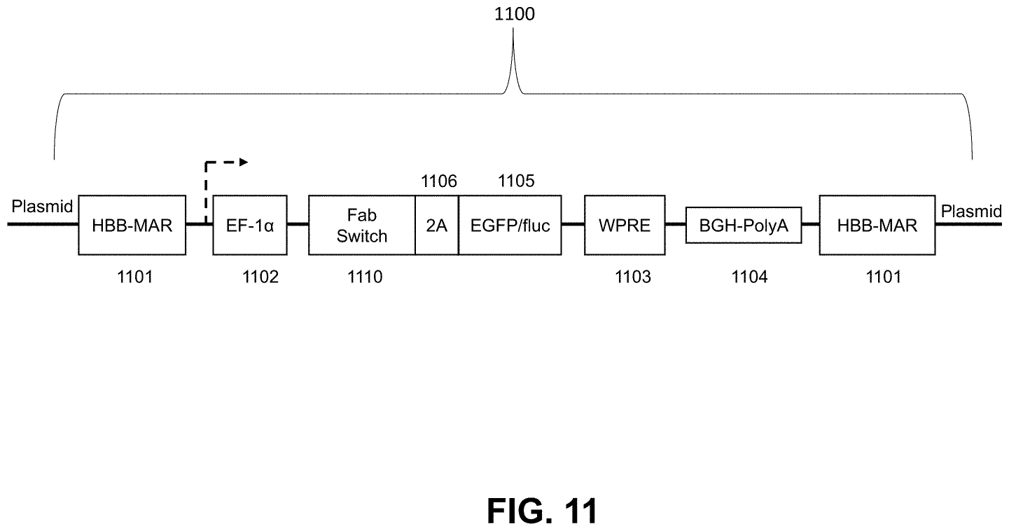

FIG. 11 shows a gene cassette 1100 comprising a Fab fragment gene 1110 under the control of an EF-1α promoter 1102. In particular embodiments, the gene cassette 1100 further comprises an MAR 1101, a WPRE 1103, and a polyadenylation sequence such as the BGH-polyA 1104. The gene cassette 1100 can also include a reporter gene such as Enhanced Green Fluorescent Protein (EGFP) 1105 under the control of a promoter 1106 such as the 2A promoter.

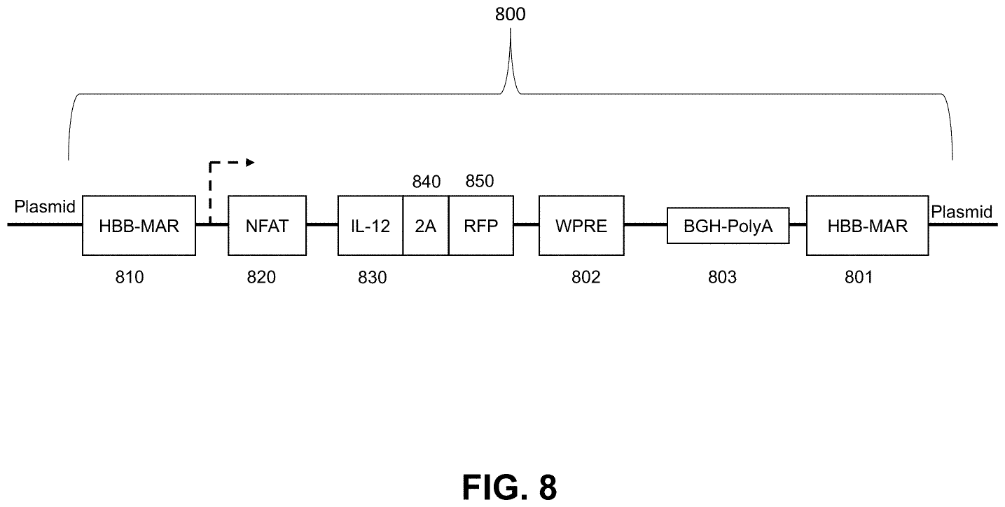

In some embodiments, the CAR cassette and the Fab fragment cassette are co-expressed Cas9/PD1, Cas9/CTL4A, and IL-12 cassettes (see FIGS. 8, 9, and 10). FIG. 8 shows the IL-12 gene cassette 800 comprising the gene for IL-12 (SEQ ID NO. 11). The gene cassette 800 further comprises an MAR sequence 810 and an NFAT promoter sequence 820. As shown in FIG. 8, the IL-12 gene 830 is fused upstream from the 2A promoter 840 and the red fluorescent protein (RFP reporter gene) 850 (see, e.g., Shun et al. (2018) Animal Model Exp. Med. 1(1):29-35). In some embodiments, the gene cassette 800 further comprises an MAR 801 downstream of the IL-12 gene, a WPRE 802, and a polyadenylation sequence such as the BGH-polyA 803.

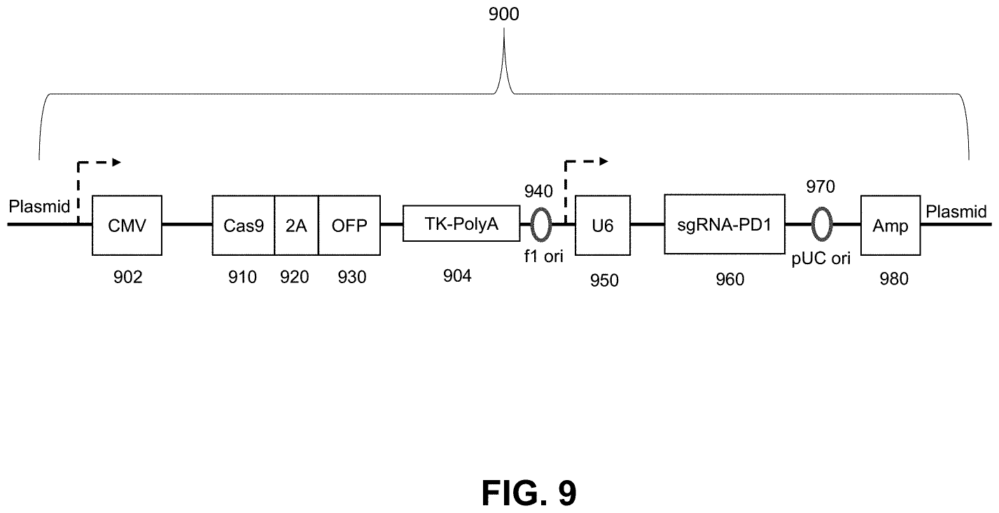

FIG. 9 shows a Cas9/PD1 gene cassette 900. The Cas9/PD1 gene 910 is under the control of a CMV promoter 902. As shown in FIG. 9, downstream of the Cas9/PD1 gene 910 is a 2A promoter 920 that controls a reporter gene such as Orange Fluorescent Reporter gene 930 (see, e.g., thermofisher.mediaroom.com/2014-09-08-Life-Technologies-Releases-CRISPR-Products -for-Simple-Rapid-Gene-Editing). In addition, FIG. 6 shows the U6 promoter sequence 950 that controls expression of PD1 gene 960. The U6 promoter sequence is shown in SEQ ID NO. 12. In some embodiments, the gene cassette 900 further comprises a polyadenylation sequence such as the TK-polyA 904. In other embodiments, the minicircle DNA includes an F1 origin 940, pUC origin 970, and resistance genes 980.

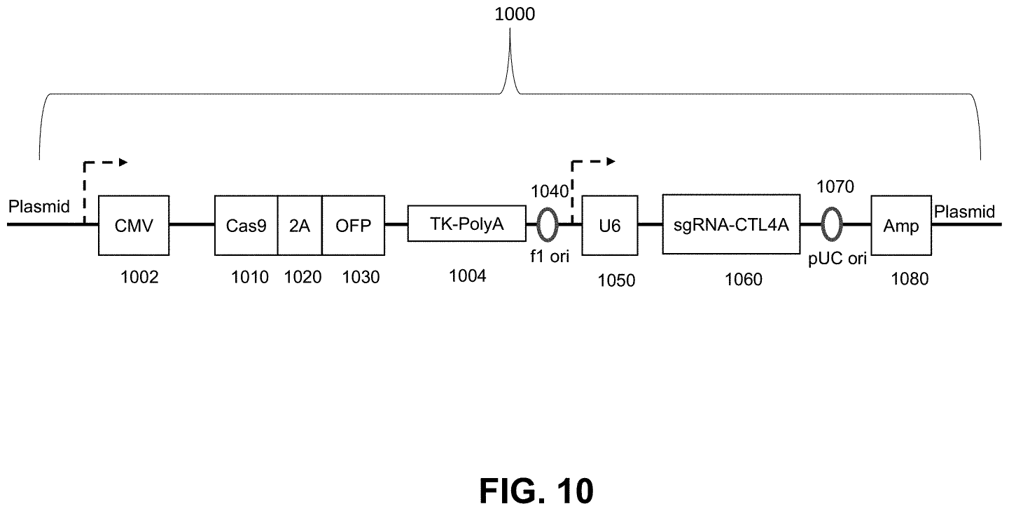

FIG. 10 shows a Cas9/CTL4A gene cassette 1000. The Cas9/CTL4A gene 1010 is under the control of a CMV promoter 1002. As shown in FIG. 9, downstream of the Cas9/CTL4A gene 1010 is a 2A promoter 1020 that controls a reporter gene such as Orange Fluorescent Reporter gene 1030 (see, e.g., thermofisher.mediaroom.com/2014-09-08-Life-Technologies -Releases-CRISPR-Products-for-Simple-Rapid-Gene-Editing). In addition, FIG. 6 shows the U6 promoter sequence 1050 that controls expression of PD1 gene 1060. In some embodiments, the gene cassette 1000 further comprises a polyadenylation sequence such as the TK-polyA 1004. As shown in FIG. 10, the minicircle DNA sequence includes an F1 origin 1040, pUC origin 1070, and resistance genes 1080.

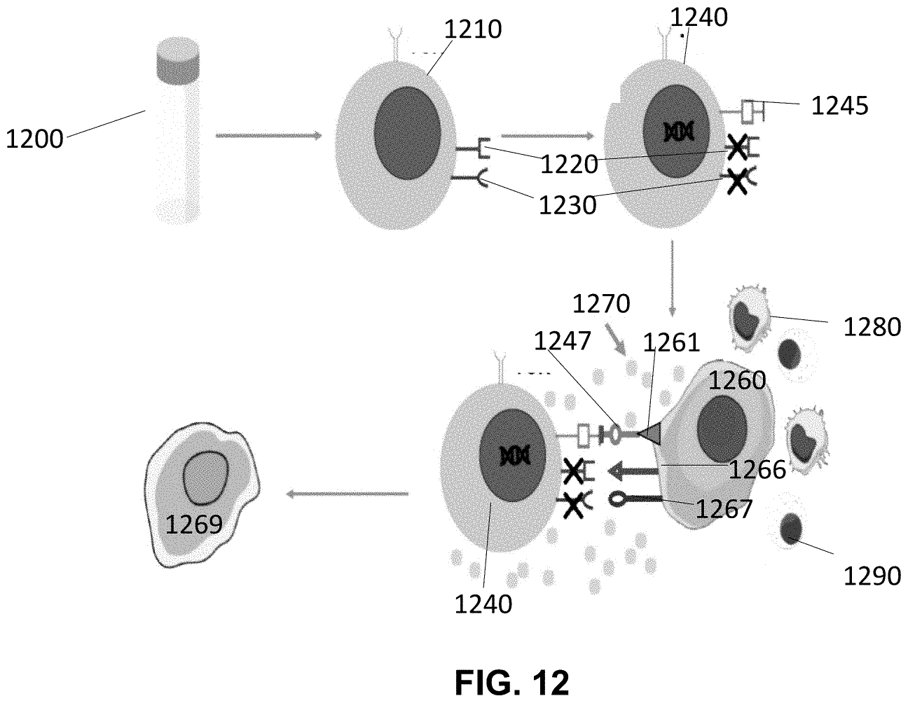

Aspects disclosed herein include methods of treating disease by killing cells associated with the disease. FIG. 12 shows an example of how the methods kill target cells. A nanoparticle formulation described herein is administered to a subject 1200. The nanoparticle formulation comprises the gene cassettes shown in FIGS. 6-11. The subject (not shown) is administered the treatment intravenously. Standard buffers for intravenous administration include normal saline, 5% dextrose, lactic acid, citric acid, acetic acid, sodium bicarbonate, maleic acid, sodium phosphate, and combinations thereof.

Returning to FIG. 12, after administration, the nanoparticle comprising an exterior with a target ligand transforms T cell 1210. The nanoparticle interior is brought into the cell in an endosome and released into the interior of the cell. The polymer decorated with peptides comprising an NLS signal facilitates the delivery of the minicircle DNA to the nucleus. There, the transposase gene cassettes of FIGS. 6, 7, and 10 into the genome of the cell. The gene cassettes encoding Cas9/PD1 and Cas9/CTL4A produce the Cas9 endonuclease and deactivate the PD1 and CTL4A receptors, respectively. This can be seen in FIG. 12 at 1220 and 1230, respectively. In addition, the T cell begins expressing the CAR and Fab cassettes (CAR/TRUCK). This leads to the “reprogrammed” T cell 1240. Upon reprogramming, T cell 1240 expresses on its cell surface the CAR 1245 and to express the Fab 1247 targeting a cell surface antigen 1261 on a target cell 1260. T cell 1240 will then identify a target cell with the Fab 1247 bound to the surface antigen 1261, in this case, a target cell 1260 through binding of the Fab 1247 to the cell surface antigen 1261 on the tumor cell 1260. The tumor cell is unable to downregulate the T cell through its CD80 1266 and PD-L1 1267 due to the lack of PD1 and CTL4A on the surface of T cell 1240. In addition, the CAR 1245 activates the IL-12 gene cassette to produce IL-12 1270, which is secreted from T cell 1240. IL-12 1270 stimulates macrophage 1280 and natural killer cell 1290 to eliminate the tumor cell 1260 (shown as dead tumor cell 1269).

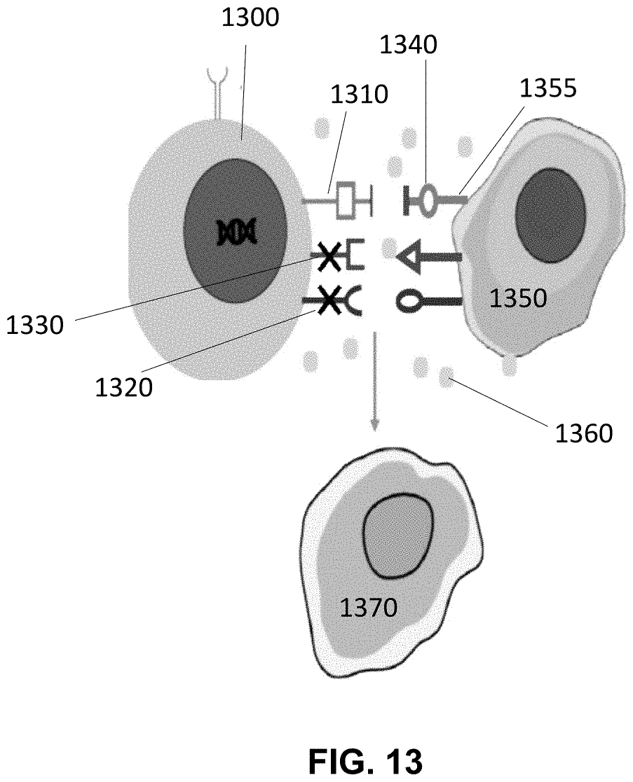

FIG. 13 shows a detailed example of how the reprogrammed T cells described herein. The reprogrammed T cell 1300. The T cell 1300 has been transformed with the plasmids of FIGS. 6-11. The T cell 1300 produces CAR 1310 on its surface and receptors PD1 1320 and CTL4A 1330 have been eliminated from the T cell. The Fab 1340 has been produced by the T cell 1300 and has associated with antigen 1355 on the surface of tumor cell 1350. In this embodiment, the Fab fragment 1340 is a polyspecific antibody associated with antigen 1355. The CAR 1310 recognizes the Fab fragment 1340 and the interaction induces expression of IL-12 1360, which will attract macrophages and natural killer cells to kill tumor cell 1350 (shown as dead tumor cell 1370).

It should be noted that all sequences disclosed herein can be deviated from so long as they continue their functionality. In particular, wobble base pairing between mRNA and tRNA allows for flexibility at the third position of codons. As such, deviations in sequences disclosed herein at this third position can yield identical amino acid sequences. In addition, there are multiple codons for most amino acids and such differences in codon sequences would yield the same amino acid sequence. It should be noted that the nucleic acid sequences disclosed herein can deviate as much as 10% or more from the sequences disclosed herein. Thus, the nucleic acid sequences can be 80%, 90%, 95%, 99% or 100% homologous to the sequences disclosed herein and still retain the required functionality.

In particular embodiments, the nanoparticles are lyophilized for storage. Prior to administration (e.g., intravenous, intramuscular, or subcutaneous) to a subject, the nanoparticles are reconstituted in an appropriate buffer for administration to a patient. As used herein, “effective amount” means the amount of a formulation necessary to produce a desired effect. The effective amount of nanoparticles for a subject will depend on the cancer type, the stage of the cancer, the weight of the subject, and the immune status of the subject. One of ordinary skill in the art would be able to determine the effective amount on a case-by-case basis. Nevertheless, the effective amount of nanoparticles will typically be in the range of about 100 μg to about 2,000 μg. In some embodiments, the effective amount is from about 50 μg to about 1,500 μg. In other embodiments, the effective amount is from about 150 μg to about 1,000 μg. In still other embodiments, the effective amount is from about 250 μg to about 750 μg.

In certain embodiments, the nanoparticle size range is from about 100 nm to about 500 nm. In particular embodiments, the nanoparticle size range is from about 50 nm to about 250 nm. In more particular embodiments, the nanoparticle size range is from about 30 nm to about 150 nm. In some embodiments, the nanoparticle size range is less than or equal to about 1 micron.

The disclosed nanoparticles can treat, and the disclosed methods provide for treatment, of cancer, including, but not limited to, neoplasms, tumors, metastases, or any disease or disorder characterized by uncontrolled cell growth, and particularly multidrug resistant forms thereof by the administration of therapeutically or prophylactically effective amounts of nanoparticles. Examples of types of cancer and proliferative disorders to be treated with the nanoparticles disclosed herein include, but are not limited to, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, mycosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. In a particular embodiment, the nanoparticles are administered to men with prostate cancer (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, and a typical prostatic stromal lesions). The treatment of cancer includes, but is not limited to, alleviating symptoms associated with cancer, the inhibition of the progression of cancer, and the promotion of the regression of cancer, and the promotion of an immune response as described herein.

The disclosed nanoparticles can be administered alone or in combination with other types of cancer treatments (e.g., radiation therapy, chemotherapy, hormonal therapy, immunotherapy, and anti-tumor agents). Examples of anti-tumor agents include, but are not limited to, cisplatin, ifosfamide, paclitaxel, taxanes, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, and taxol. In some embodiments, the disclosed nanoparticles are administered after surgical resection of cancer. In another embodiment, the nanoparticles are administered to an animal, preferably a mammal and most preferably a human, in conjunction with chemotherapy or radiotherapy. The nanoparticles and methods disclosed herein can be used for the treatment of microbial infections in an animal, preferably a mammal and most preferably a human, said methods comprising the administration of a therapeutically or prophylactically effective amount of nanoparticles to the subject. Examples of microbial infections which can be treated include yeast infections, fungal infections, protozoan infections, and bacterial infections. Bacteria which cause microbial infections include, but are not limited to, Streptococcus pyogenes, Streptococcus pneumoniae, Neisseria gonorrhoeae, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazekii, Rickettsia tsutsugumushi, Chlamydia spp., and Helicobacter pylori.

### EXAMPLES

Polyethylenimine (PEI) nanocomplex:

Step 1:

PEI is commercially available branched form polyethylenimine (bPEI) (25 kDa), will be diluted to 10% w/v then further will be neutralized to stock solutions of 5 mg/ml (0.2 mM) in PBS (10 mM Na2HPO4, 2 mM KH2PO4, 3 mM KCl, 140 mM NaCl; pH 7.4) and 5 mg/ml neutralized with HCl. N-succinimidyl 3-(2-Pyridyldithio)propionic acid (SPDP) and N-succinimidyl 4-(Maleimidomethyl)cyclohexanecarboxylate (SMCC) for amine and cysteine bifunctional linkers.

For reactions SPDP and SMCC will be dissolved in dimethyl sulfoxide (DMSO) to 40 mM or 20 mM immediately before use or stored at −80° C. in DMSO which will be retained N-hydroxysuccinimidyl (NHS) reactivity for at least a few weeks.

Step 2:

Activation of PEI 19.2 mg (5.65 Amol) of bifunctional PEG (3.4 kDa), containing both an a-vinyl sulfone and an N-N hydroxysuccinimide ester group, was weighed into a glass flask. 4.293 ml of a PEI solution (corresponding to 12.15 mg/0.486 Amol PEI; 282.4 Amol total amines) in 0.1 M borate buffer at pH 5.5 will be added and stirred. The activation reaction will be carried out for 4 h at room temperature, followed by pH adjustment to 7 with 1 N NaOH and additionally incubate for 2 h at room temperature.

Step 3:

The TAT-PEG-PEI conjugate is composed of a 25 kDa PEI core, a 3.4 kDa PEG linker and an oligopeptide sequence.

TAT (amino acids sequence 49-57 (SEQ ID NO. 9) RKKRRQRRR and amino acids 48-60 (SEQ ID NO. 10) GRKKRRQRRRPPQ of the HIV TAT protein). The nuclear localization signal (NLS, with the sequence (SEQ ID NO. 11) VQRKRQKLMP/SKKKKIKV/GRKRKKRT) oligopeptide roughly 2.98 mg will be dissolved in 866 Al pure water then couple with activated PEG-PEI yielding approximately 1% based upon amine functions. The mixture will be further stirred for additional 2 h at room temperature and stirring at 4° C. overnight in the dark.

The two-step reaction:

Bifunctional NHS PEG will be used to activate PEI via the primary amine reactive N-N-hydroxysuccinimide ester moiety at pH 5.5, thus avoiding the conjugation and cross linking of the a-vinyl sulfone groups to the amine functions of PEI, which occurs at higher pH.

Reaction will be carried out for 4 h at pH 5.5. After adjusting the pH to 7, the solution will be stirred for additional 2 h to allow hydrolysis of eventually unreacted reagent. Thus, after activation of the PEI core, the oligopeptide containing a cysteine moiety at the C terminus will be coupled to the a-vinyl sulfone group at pH 7. The composition of the conjugates will be calculated by assuming that PEI-TAT-NLS conjugation will be completed.

Step 4:

The nano polyplexes consisting of plasmid DNA of CAR TRUCK/Cas9/PD1-CTL4A will be prepared in sterile isotonic glucose solution at pH 7.4. Briefly, the PEI-TAT-NLS polymer solution will be added rapidly to the DNA and mix by vigorous pipetting followed by 10-20 min incubation at room temperature prior to use. When various polymer nitrogen to DNA phosphate ratios (N/P) will be investigated, the concentration of the PEI or TAT-NLS-PEG-PEI solution will be adjusted to the amount of DNA (20 Ag/ml polyplex solution for the prostate cell lines experiments and 10 fold increase for in vivo xenograft prostate animal experiments) in order to maintain N/P ratios between 0.5 and 10.

Our proposed nanopolyplex particle charge, size and aggregation tendency and surface charges will be determined by measuring the zeta-potential, dynamic light scattering, Atomic force microscopy, Transmission Electron Microscopy, Complex size measurements, viscosity, refractive index will be determined.

Step 5:

PEI-plasmid minicircle DNA nanopolyplexes further incorporating a negatively-charged poly-γ-glutamic acid (PGA) targeting anti-CD3 and or CD19 or CD22 by double emulsion solvent evaporation technique by adding aqueous solution of BSA (2 mg) and 250 μg PEI nanopolyplexes will be emulsified at 0° C. for 30 sec and subsequently, the mixture will be emulsified in 1.5 mL of 1% w/v PVA solution at 0° C. for two min. The emulsion will be poured into 25 mL 0/3% PVA solution under moderate magnetic stirring. In order to remove chloroform, the emulsion will be stirred at high speed for three hours at room temperature. Nanoparticles will be collected by centrifugation at 16602×g for 2 h, following washing for three times with sterile distilled water to remove unentrapped PVA, PEI, DNA, and BSA, then the nanoparticles will be lyophilized or nanoprecipitation via mixing PEI/plasmid DNA/CAR TRUCK/Cas9/PD1-CTL4A/PGA (combo delivery nanoparticles-CDNPs) with specific ratio (N/P/C ratio) and the particles present positive surface charge by mixing the PEI with plasmid DNA/CAR TRUCK/Cas9/PD1-CTL4A, and then the resulting complexes will be vortexed again with PGA-solution to form a combo delivery nanoparticles-CDNPs as a spherical nanoparticles with about 200 nm diameter nanopolyplexes. All the routine nanocharacterization will be followed similar to step 4.

Administering nanoparticles to a subject:

To assess CAR T cell nano mediated tumor clearance in vivo in a xenograft model: PC-3 cells (3×10⁶) will be suspended in 100 μL of phosphate buffered saline and inject subcutaneously into the flank of 4-week-old nude mice. Two weeks later, when the tumors reaches a volume of 100 mm3, the mice will be divided randomly into 4 groups of 6 mice each. An intratumor injection of CAR TRUCK/Cas9/PD1-CTL4A/PGA (combo delivery nanoparticles-CDNPs) (10 μM×50 μL) of phosphate buffered saline will be administrated every week along with controls. The tumor volumes will be calculated using the formula: length×width²×0.5.

Although the invention has been described by reference to specific embodiments, it should be understood that numerous changes may be made within the spirit and scope of the inventive concepts described. Accordingly, it is intended that the invention is not limited to the described embodiments, but that it has the full scope defined by the language of the following claims.

### FURTHER EXAMPLES

Anti-V5 variable light and heavy chains:

VL and VH against prostate specific antigen from EpiVax and/or Add gene

CD8 alpha hinge regions:
SEQ ID NO. 1:
TTTPAPRPPTPAPTIASQPLSLRPEACRPAAGGAVHTRGLDFACD

CD28 hinge region:

Accession number AAF03881

4-1BB:

Accession number U03397

CD3ζ:

Accession number AY839629

V5 tag:
SEQ ID NO. 2:
GKPIPNPLLGLDST

Common Epitope Tag that recognizes a 14 amino acid sequence (SEQ ID NO. 3: GKPIPNPLLGLDST) derived from the P and V proteins of the paramyxo virus simian virus (J Gen Virol. 1987 Nov;68 (Pt 11):2769-80)

MAR:

HBB-MAR—Accession number NM_000518.5/NM_003073.5/U88348

SEQ ID NO. 4: BGH-polyA sequence:
CTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAAT
GAGGAAATTGCATCGCATTGTCTGAGTAGGTGTCATTCTATTCTGGGGG
GTGGGGTGGGGCAGGACAGCAAGGGGGAGGATTGGGAAGACAATAGCAG
GCATGCTGGGGATGCGGTGGGCTCTATGG-

this can be purchased from addgene or vector builder.

Accession number E16476

m194-1BBz CAR:

Accession: U03397.1

Utilizing 2A peptide sequence for m194-1BBz.

iPB7:

Accession number: EF587698

The plasmid encodes the hyperactive iPB7 piggyBac transposase under the control of the EF1alpha promoter.

Cas9:

Cas9 protein purchased from PNA Bio [www.pnabio.com]

anti-CD3, anti-CD19, and anti-CD22:

Switch molecules obtained from EpiVax or Add gene. Below example sequences.

SEQ ID NO. 5: Anti-CD19 Light chain WT:
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO. 6: Anti-CD19 Light chain LCC1:
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEIKRTVAAPSVFIFPPSDEQLKSGTASVVCLLNNFYPREAKVQ
WKVDNALQSGNSQESVTEQDSGGGGSNYHLENEVARLKKLGGGGSDSTY
SLSSTLTLSKADYEKHKVYACEVTHOGLSSPVTKSFNRGEC
SEQ ID NO. 7: Anti-CD19 Heavy chain (Fab) WT:
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSC
SEQ ID NO. 8: Anti-CD19 Heavy chain (Fab) HCC1:
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSSASTKGPSVFPLAPSSKSTSGGTAALGC
LVKDYFPEPVTVSWNSGALTSGVHTFPAVLQSGGGGSNYHLENEVARLK
KLGGGGSLYSLSSVVTVPSSSLGTQTYICNVNHKPSNTKVDKKVEPKSC

1-79. (canceled)

80. A nanoparticle comprising an interior portion comprising one or more DNA molecules and a polymer bound to a protein, the protein comprising a first amino acid sequence directing the protein to the nucleus of a cell and a second sequence, wherein the one or more DNA molecules are associated with the polymer by way of opposing charges.

81. The nanoparticle of claim 80, wherein the protein further comprises a second sequence selected from the group consisting of a TAT sequence and a MLS sequence.

82. The nanoparticle of claim 80, the polymer is selected from the group consisting of poly-β-amino ester, polyethylenimine, pentaethylenehexamine, polyethylene glycol, polyvinyl alcohol, poly(lactide-co-glycolide, poloxamer, poly(N-vinylpyrrolidone, gelatin, human albumin, and starch.

83. The nanoparticle of claim 80, wherein the DNA molecule is a minicircle plasmid.

84. The nanoparticle of claim 80, wherein the DNA molecules comprise two or more genes selected from the group consisting of a CAR gene, an IL-12 protein, a first Cas9 endonuclease modified to cleave a DNA sequence in the PD1 gene to inactivate the PD1 gene, a second Cas9 endonuclease modified to cleave a DNA sequence in the CTL4A gene to inactivate the CTL4A gene, a targeting component to target a cell, and a CD3-theta domain.

85. The nanoparticle of claim 84, wherein the CAR gene encodes a molecule comprising a transmembrane domain, one or more cytoplasmic domains, and a targeting ligand selected from the group consisting of a variable heavy chain and a variable light chain of a Fab fragment.

86. The nanoparticle of claim 84, wherein the targeting component is selected from the group consisting of recombinant antibodies, monoclonal antibodies, Fab fragments, Fab2 fragments, single chain variable fragments, diabodies, and receptor ligands.

87. The nanoparticle of claim 84, wherein the targeting component is a Fab fragment comprising a tag and wherein the one or more DNA molecules comprise a first gene that encodes a CAR gene.

88. The nanoparticle of claim 80, further comprising an exterior selected from the group consisting of a polyglutamic acid polymer and a targeting group selected from the group consisting of recombinant antibodies, monoclonal antibodies, Fab fragments, Fab2 fragments, single chain variable fragments, diabodies, and receptor ligands.

89. The nanoparticle of claim 80, wherein the nanoparticle is lyophilized.

90. A method of treating a condition in a subject, the method comprising:
administering a nanoparticle to the subject, the nanoparticle comprising an interior portion comprising one or more DNA molecules and a polymer bound to a protein, the protein comprising a first amino acid sequence directing the protein to the nucleus of a cell and a second sequence, wherein the one or more DNA molecules are associated with the polymer by way of opposing charges, wherein the nanoparticles reprogram a plurality of T cells in the subject; and
targeting, through the T cells, a plurality of cells causing the condition in the subject,
wherein the T cells instigate the death of the plurality of cells causing the condition, thereby treating the disease.

91. The method of claim 90, wherein the protein further comprises a second sequence selected from the group consisting of a TAT sequence and a MLS sequence.

92. The method of claim 90, wherein the polymer is selected from the group consisting of poly-β-amino ester, polyethylenimine, pentaethylenehexamine, polyethylene glycol, polyvinyl alcohol, poly(lactide-co-glycolide, poloxamer, poly(N-vinylpyrrolidone, gelatin, human albumin, and starch.

93. The method of claim 90, wherein the DNA molecule is a minicircle plasmid.

94. The method of claim 90, wherein the DNA molecules comprise two or more genes selected from the group consisting of a CAR gene, an IL-12 protein, a first Cas9 endonuclease modified to cleave a DNA sequence in the PD1 gene to inactivate the PD1 gene, a second Cas9 endonuclease modified to cleave a DNA sequence in the CTL4A gene to inactivate the CTL4A gene, a targeting component to target a cell, or a CD3-theta domain.

95. The method of claim 94, wherein the CAR gene encodes a molecule comprising a transmembrane domain, one or more cytoplasmic domains, and a targeting ligand selected from the group consisting of a variable heavy chain and a variable light chain of a Fab fragment.

96. The method of claim 94, wherein the targeting component is selected from the group consisting of recombinant antibodies, monoclonal antibodies, Fab fragments, Fab2 fragments, single chain variable fragments, diabodies, and receptor ligands.

97. The method of claim 94, wherein the targeting component is a Fab fragment comprising a tag and wherein the one or more DNA molecules comprise a first gene that encodes a CAR gene.

98. The method of claim 90, further comprising an exterior selected from the group consisting of a polyglutamic acid polymer and a targeting group selected from the group consisting of recombinant antibodies, monoclonal antibodies, Fab fragments, Fab2 fragments, single chain variable fragments, diabodies, and receptor ligands.

99. The method of claim 90, wherein the nanoparticle is lyophilized.
